(19) United States
(12) Patent Application Publication
(10) Pub. No.: US 2007/0031363 A1
(43) Pub. Date: Feb. 8, 2007

(54) Epidermal exfoliation method using a topical composition containing vegetable oil, sugar, vinegar, and egg yolk

(76) Inventor: Diane Simpson Bunker, Orem, UT (US)

Correspondence Address: Angus C. Fox, III, 4093 N. Imperial Way, Provo, UT, 84604-5386, US

(21) Appl. No.: 11/582,258

(22) Filed: Oct. 15, 2006

Related U.S. Application Data
(63) Continuation-in-part of application No. 10/871,104, filed on Jun. 18, 2004.

(51) Int. Cl. A61K 8/73

(52) U.S. Cl. 424/70.13; 424/401

(57) ABSTRACT

A method for exfoliating an epidermal skin layer comprising the steps of: applying a dermatological composition to an epidermal layer to be exfoliated, the dermatological composition containing vegetable oil; at least one sugar; dilute acetic acid; and egg yolk; allowing the composition to remain on the area to be exfoliated for a period of time which will allow the composition to be at least partially absorbed by the epidermal skin layer; and initiating a bidirectional rubbing motion with the hands on the area to be exfoliated in order to remove dead epidermal cells as particulate debris.

This is a continuation-in-part of application Ser. No. 10/871,104, which was filed on Jun. 18, 2004, and which is now abandoned.

### BACKGROUND OF THE INVENTION

1. Field of the Invention

This invention relates to methods for exfoliating dermatological compositions for topical application that are useful for promoting skin exfoliation and moisturization and, more particularly, to such compositions containing dilute carboxylic acids.

1. Description of the Prior Art

Vinegar, which is dilute acetic acid, has been used as a health and beauty aid for thousands of years. Hippocrates, the Greek physician, extolled the therapeutic qualities of vinegar. Helen of Troy reputedly bathed in vinegar. Before robbing those who had died of the black plague, thieves washed themselves with vinegar to avoid contracting the disease. Although printed publications indicate that dermatological compositions containing vinegar have been known for at least 35 years, such compositions have probably been used for a century or more.

In the book Swedish Beauty Secrets (Health Plus Publishers, 1971), Paavo 0. Airola discloses a “Formula F Cream”, which he claims softens and moisturizes the skin. The Formula F Cream formulation is as follows: ½ cup sesame oil, ¼ cup avocado oil, ¼ cup almond oil, 2 fresh egg yolks, and 1 tsp. apple cider vinegar.

In Kitty Campion's Book of Herbal Beauty (Random House, 1980), a skin softener, lubricant and lotion titled “Mayonnaise Skin Food” is disclosed. The formulation includes: 1 large raw egg yolk, 4 tbsp. cider vinegar, ½ cup corn oil, 3 tbsp. wheatgerm oil, 4 tsp. double-strength decoction of marshmallow root, and 1 tsp. clear honey.

U.S. Pat. No. 4,255,418 to Florence H. Bailey discloses an ANTI-ACNE LOTION which is formulated using zinc oxide, a sodium borate, vinegar, oils, cucumber juice and hydroxybenzenes.

U.S. Pat. No. 5,006,557 to Yee Siu-Ming discloses an ACNE SOLUTION including glacial acetic acid, almond essence, glycerine and water.

It is interesting to note that both of the anti-acne formulations cited above include vinegar as a primary ingredient. Acne is a skin condition caused by overactivity of the sebaceous glands that secrete oily substances through the hair follicles on to the skin.

The blackheads and pimples, which are characteristic of acne, usually occur on the face, where the greatest number of these glands exist. At puberty, the male hormone testosterone—which is produced naturally in both men and women—causes the sebaceous glands to produce more of an oily substance called sebum, thus making the skin more greasy. The production of excess sebum can result in the clogging of hair follicles (also known as pores). A bacterium normally present on the skin, known as propionobacterium acnes, thrives within the clogged follicles. This bacterial infection causes inflammation, which is responsible for the redness and swelling that are symptomatic of acne. In order to alleviate these symptoms, clogging of the hair follicles must be prevented and the bacterial infection must be abated. It seems quite clear that skin formulations containing acetic acid are effective on both counts. It is both a mild bactericide and a skin exfoliant. As dead layers of skin are removed, the sebum clogging the upper portion of the hair follicles is also removed.

U.S. Pat. No. 5,560,916 to Constantin Koulbanis discloses a COSMETIC COMPOSITION CONTAINING VINEGAR AS ACTIVE ANTI-AGEING AGENT, AND ITS USE IN THE TREATMENT OF DERMATOLOGICAL AGEING.

U.S. Pat. No. 6,153,649 to Jean Malgnan discloses the USE OF CARBOXYLIC ACIDS HAVING SULPHUR FUNCTION FOR PROMOTING SKIN EXFOLIATION OR STIMULATING EPIDERMAL REGENERATION. The product is claimed to be useful for controlling intrinsic and extrinsic skin ageing, and as a non-therapeutic skin exfoliation and regeneration agent.

### SUMMARY OF THE INVENTION

A first object of the present invention is to provide a cosmetic or dermatological composition for use in promoting the exfoliation of dead skin and the moisturization and sterilization of the underlying dermal layers which are exposed by the exfoliative action. A second object of the invention is to provide a method for using the cosmetic or dermatological composition so provided.

The cosmetic or dermatological composition is formulated with the following primary ingredients: vegetable oil, in an amount within a range from about 35% to about 50% of total weight; at least one sugar, in an amount within a range from about 15% to about 30% of total weight; acetic acid, in an amount within a range from about 15% to about 20% of total weight; egg yolk, in a dehydrated powder equivalent amount, within a range from about 2.7% to about 4.3% of total weight; a thickener, in an amount within a range from about 1% to 3% of total weight; and an emulsifier, in an amount within a range from about 0.25% to about 1% of total weight. Optional secondary ingredients may include water, in an amount up to about 7% of total weight; alpha tocopheryl acetate (vitamin E) in an amount of up to 2 international units per gram of total weight; and at least one natural or artificial fragrance in an amount within a range from about 0.1% to about 0.5% of total weight.

The thickener is selected from the group consisting of modified food starch, agar and silica gel; the emulsifier is selected from the group consisting of lecithin, carrageenin, and lactose; at least one sugar is selected from the group consisting of dextrose, fructose, glucose, honey, invert sugar, lactose, maltose, and sucrose; acetic acid is at least one vinegar selected from the group consisting of apple cider vinegar, wine vinegar, white vinegar, fruit vinegar, malt vinegar, rice vinegar, cane vinegar and corn sugar vinegar; and vegetable oil is selected from the group consisting of soybean oil, flax seed oil, sesame seed oil, sunflower oil, palm oil, palm kernel oil, avocado oil, olive oil, corn oil, safflower oil, peanut oil, canola oil, and grape seed oil. For a presently preferred embodiment of the invention, modified food starch is selected as the thickener, lecithin is selected as the emulsifier, sucrose is selected as the sugar; white distilled vinegar is selected as the dilute acetic acid; soybean oil is selected as the vegetable oil. The egg yolk component can be provided as either a dehydrated powder or as an equivalent amount of fresh egg yolk.

The dermatological composition heretofore described is preferably used on dry (non-hydrated) skin. It may be used over makeup and should be used before washing or showering. For facial conditioning, about 5 grams of the composition is rubbed onto the palms and fingers of dry hands, then quickly spread in a thin layer over the face, paying close attention to areas around the eyes, lips, nose, under the chin and neck. After facial application, the hands are washed with soap and water, then towel dried. When the composition has remained on the face for about 10 minutes, a bidirectional rubbing motion is begun with the hands on the face. The uppermost layers of the epidermis will be removed and fall away. The rubbing is repeated at different angles for all regions of the face treated with the composition. If the skin is loose, it may be tightened during the rubbing step with one hand while the other does the rubbing. Alternately, the skin may be tightened with the facial muscles. The total time required to exfoliate the face using the disclosed composition and rubbing process takes from 20 to 30 minutes. In humid climates where skin is naturally moist and hydrated, it will be necessary to re-apply the composition about ten minutes after the initial application and waiting another ten minutes thereafter before beginning the rubbing motion. The product may also be used to promote skin exfoliation on other parts of the body. For these areas, a double layer application is advised due to the varied skin textures which may be present. Once applied, the skin must be firmly rubbed for several minutes before exfoliation begins.

### DETAILED DESCRIPTION OF THE INVENTION

The cosmetic or dermatological composition for use in promoting the exfoliation of dead skin and the moisturization of and reduction of bacteria count on underlying dermal layers which are exposed by the exfoliative action will now be disclosed in detail.

Vegetable oil is a primary ingredient of the dermatological composition which is a subject of this invention, comprising between about 35 percent and about 50 percent thereof, by weight. The vegetable oil is selected from the group consisting of soybean oil, flax seed oil, sesame seed oil, sunflower oil, palm oil, palm kernel oil, avocado oil, olive oil, corn oil, safflower oil, peanut oil, canola oil, grape seed oil, and mixtures thereof. For a presently preferred embodiment of the invention, cold pressed soybean oil is used exclusively.

Sugar is another primary ingredient of the composition, comprising between about 15 percent and about 20 percent thereof, by weight. The sugar is selected from the group consisting of dextrose, fructose, glucose, honey, invert sugar, lactose, maltose, sucrose, and mixtures thereof. For a presently preferred embodiment of the invention, sucrose is used exclusively.

Dilute acetic acid having a concentration of between about 4 to 8 percent, by weight, is another primary ingredient of the composition, comprising between about 15 percent and about 30 percent thereof, by weight. The glacial acetic acid equivalent would, therefore, be within a range from about 0.6 percent to about 2.4 percent of total composition weight. Dilute acetic acid is most readily available as vinegar, which may be selected from the group consisting of apple cider vinegar, wine vinegar, white vinegar, fruit vinegar, malt vinegar, rice vinegar, cane vinegar, corn sugar vinegar, and mixtures thereof. For a presently preferred embodiment of the invention, white distilled vinegar, manufactured from grain alcohol, is used exclusively, because of its mild odor.

Egg yolk is another primary ingredient of the composition, comprising between about 2.7 percent and about 4.3 percent thereof, by weight. The egg yolk weight is taken as a dehydrated powder equivalent. Thus, fresh egg yolk may be substituted dehydrated powdered egg yolk, with one medium sized egg yolk being roughly equivalent to 7 grams of powdered egg yolk.

A thickener is another primary ingredient of the composition, comprising between about 1 percent and about 3 percent thereof, by weight. The thickener is selected from the group consisting of modified food starch, agar, silica gel, and mixtures thereof.

An emulsifier is another primary ingredient of the composition, comprising between about 0.25 percent and about 1 percent thereof, by weight. The emulsifier is selected from the group consisting of lecithin, carrageenin, lactose, and mixtures thereof.

Optional secondary ingredients may include water, in an amount up to about 7 percent of total weight; alpha tocopheryl acetate (vitamin E) in an amount of up to 2 international units per gram of total weight; and at least one natural or artificial fragrance in an amount within a range from about 0.1 percent to about 0.5 percent of total weight.

The manufacture of the presently preferred embodiment of the cosmetic and dermatological composition will now be described in detail. As the formula is sensitive to high humidity, it is preferred to manufacture the composition in an environment where the relative humidity is within a range of about 15 to 25 percent and the temperature is within a range of about 20 to 22° C.

1. Refrigerated egg yolk powder of the required amount is allowed to warm to ambient temperature;

2. The required amount of lecithin is heated to about 50° C.;

3. The lecithin is stirred into the required amount of vegetable oil;

4. As an option, alpha tocopheryl acetate (vitamin E) is stirred into the lecithin/vegetable oil mixture;

5. The egg yolk powder is placed in a blender, along with the required quantity of sugar;

6. The sugar and egg yolk powder are dry mixed, preferably by repeatedly pulsing the blender;

7. The required amount of dilute acetic acid is added to the dry mixture;

8. The sugar, egg yolk powder and dilute acetic acid are blended for about 30 to 45 seconds;

9. With the blender running continuously, one-fourth of the lecithin/vegetable oil mixture is gradually added over a period of about 1.5 minutes, to the sugar/egg yolk/dilute acetic acid mixture;

10. With the blender still running continuously, the balance of the lecithin/vegetable oil mixture is gradually added during a period of 2 to 3 minutes;

11. As a further option, fragrances can be added to the mixture with the blender still running;

12. Finally, water in the desired amount and at a temperature of about 50° C., is added to the mixture with the blender still running.

During the manufacturing process, the final product will have acquired a temperature of about 27.5° C. It should now be packaged and refrigerated at a temperature within a range of between 2.2 and 4.4° C. Average shelf life of the product is about 45 days. The average period of usability is about 60 days from the production date. Shelf life can be maximized by packaging in airtight, UV-filtered containers. Vacuum packaging of the product in foil pouches is one effective packaging method.

Use of the heretofore described cosmetic and dermatological composition will now be described in detail. The exfoliation process works most effectively when the composition is applied dry (non-hydrated) skin. It may be used over makeup and should be used before washing or showering. For facial conditioning, about 5 grams (roughly one teaspoonful) of the composition is rubbed onto the palms and fingers of dry hands, then quickly spread in a thin layer over the face, paying close attention to areas around the eyes, lips, nose, under the chin and neck. After facial application, the hands are washed with soap and water, then towel dried. When the composition has remained on the face for a period within a range of about eight to twelve minutes, a bidirectional rubbing motion is begun with the hands on the face. Particles of dead skin will be removed and fall away. The rubbing is repeated at different angles for all regions of the face treated with the composition. If the skin is loose, it may be tightened during the rubbing step with one hand while the other does the rubbing. Alternately, the skin may be tightened with the facial muscles. The total time required to exfoliate the face using the disclosed composition and rubbing process takes from 20 to 30 minutes. In humid climates where skin is naturally moist and hydrated, it will be necessary to re-apply the composition about ten minutes after the initial application and waiting another ten minutes thereafter before beginning the rubbing motion. The product may also be used to promote skin exfoliation on other parts of the body. For these areas, a double layer application is advised due to the varied skin textures which may be present. Once applied, the skin must be firmly rubbed for several minutes before exfoliation begins.

Laboratory analysis performed with a Zeiss light microscope on residue samples resulting from use of the disclosed dermatalogical composition by a 10-year-old girl, a 49-year-old male, and a 48-year-old female indicate that in all three cases, dead skin cells constituted between 30 and 50 percent of the residue samples.

As acetic acid is a well-known anti-bacteriological agent, the inclusion of that compound in the disclosed cosmetic and dermatalogical composition ensures that the composition will have significant antiseptic qualities.

Although only several embodiments of the invention have been described, it will be obvious to those having ordinary skill in the art that changes and modifications may be made thereto without departing from the scope and the spirit of the invention as hereinafter claimed.

What is claimed is:

1. A method for exfoliating an epidermal skin layer comprising the steps of:
applying a dermatological composition to the palms and fingers of dry hands, the composition containing vegetable oil, in an amount within a range from about 35% to about 50% of total weight; at least one sugar, in an amount within a range from about 15% to about 30% of total weight; dilute acetic acid having a concentration of about 4 to 8 percent, in an amount within a range from about 15% to about 20% of total weight; and egg yolk, in a dehydrated powder equivalent amount, within a range from about 2.7% to about 4.3% of total weight;
evenly applying the composition on the hands to an epidermal skin layer to be exfoliated as a thin layer of generally even thickness;
allowing the composition to remain on the area to be exfoliated for a period of time which will allow the composition to be at least partially absorbed by the epidermal skin layer;
initiating a bidirectional rubbing motion with the hands on the area to be exfoliated in order to remove dead epidermal cells as particulate debris.

2. The method of claim 1, wherein said dermatological composition is allowed to remain on the area to be exfoliated for a period within a range of about eight to 12 minutes.

3. The method of claim 1, wherein said dermatological composition further contains at least one thickener, in an amount within a range from about 1% to 3% of total weight; and at least one emulsifier, in an amount within a range from about 0.25% to about 1% of total weight.

4. The method of claim 3, wherein said at least one thickener is selected from the group consisting of modified food starch, agar and silica gel.

5. The method of claim 3, wherein said emulsifier is selected from the group consisting of lecithin, carrageenin, and lactose.

6. The method of claim 1, wherein said egg yolk is fresh.

7. The method of claim 1, wherein said at least one sugar is selected from the group consisting of dextrose, fructose, glucose, honey, invert sugar, lactose, maltose, and sucrose.

8. The method of claim 1, wherein said dilute acetic acid is at least one vinegar selected from the group consisting of apple cider vinegar, wine vinegar, white vinegar, fruit vinegar, malt vinegar, rice vinegar, cane vinegar and corn sugar vinegar.

9. The method of claim 1, wherein said at least one vegetable oil is selected from the group consisting of soybean oil, flax seed oil, sesame seed oil, sunflower oil, palm oil, palm kernel oil, avocado oil, olive oil, corn oil, safflower oil, peanut oil, canola oil, grape seed oil, and mixtures thereof.

10. The method of claim 1, wherein said dermatological composition further contains water in an amount up to about 7% of total weight.

11. A method for exfoliating an epidermal skin layer comprising the steps of:
applying a generally even layer of a dermatological composition to an epidermal layer to be exfoliated, said dermatological composition containing vegetable oil, in an amount within a range from about 35% to about 50% of total weight; at least one sugar, in an amount within a range from about 15% to about 30% of total weight; dilute acetic acid having a concentration of about 4 to 8 percent, in an amount within a range from about 15% to about 20% of total weight; and egg yolk, in a dehydrated powder equivalent amount, within a range from about 2.7% to about 4.3% of total weight;
allowing the composition to remain on the area to be exfoliated for a period of time which will allow the composition to be at least partially absorbed by the epidermal skin layer;
initiating a bidirectional rubbing motion with the hands on the area to be exfoliated in order to remove dead epidermal cells as particulate debris.

12. The method of claim 1, wherein said dermatological composition is allowed to remain on the area to be exfoliated for a period within a range of about eight to 12 minutes.

13. The method of claim 11, wherein said dermatological composition is applied in two layers sequentially, with about equal periods of time between the first application and the initiation of the bidirectional rubbing motion after the second application.

14. The method of claim 11, wherein said dermatological composition further contains at least one thickener, in an amount within a range from about 1% to 3% of total weight; and at least one emulsifier, in an amount within a range from about 0.25% to about 1% of total weight; and water in an amount up to about 7% of total weight.

15. The method of claim 14, wherein said at least one thickener is selected from the group consisting of modified food starch, agar and silica gel.

16. The method of claim 14, wherein said emulsifier is selected from the group consisting of lecithin, carrageenin, and lactose.

17. The method of claim 11, wherein said egg yolk is fresh.

18. The method of claim 11, wherein said at least one sugar is selected from the group consisting of dextrose, fructose, glucose, honey, invert sugar, lactose, maltose, and sucrose.

19. The method of claim 11, wherein said dilute acetic acid is at least one vinegar selected from the group consisting of apple cider vinegar, wine vinegar, white vinegar, fruit vinegar, malt vinegar, rice vinegar, cane vinegar and corn sugar vinegar.

20. The method of claim 11, wherein said at least one vegetable oil is selected from the group consisting of soybean oil, flax seed oil, sesame seed oil, sunflower oil, palm oil, palm kernel oil, avocado oil, olive oil, corn oil, safflower oil, peanut oil, canola oil, grape seed oil, and mixtures thereof.
